(19) United States
(12) Patent Application Publication
(10) Pub. No.: US 2004/0054287 A1
(43) Pub. Date: Mar. 18, 2004

(54) Ultrasonic imaging devices and methods of fabrication

(76) Inventor: Douglas Neil Stephens, Davis, CA (US)

Correspondence Address: FISH & NEAVE, 1251 AVENUE OF THE AMERICAS, 50TH FLOOR, NEW YORK, NY, 10020-1105, US

(21) Appl. No.: 10/233,870

(22) Filed: Aug. 29, 2002

(51) Int. Cl. A61B 8/14

(52) U.S. Cl. 600/459

(57) ABSTRACT

A sensor for an ultrasound imaging catheter and methods of fabrication are provided. The sensor may be based on a flex circuit on which a block of piezoelectric sensor array transducer material is mounted. The flex circuit may include electrical conductors that are electrically connected to electrodes on the piezoelectric blocks. A matching layer may be formed on the piezoelectric blocks between the blocks and the flex circuit substrate. Individual transducer array elements may be formed by dividing a piezoelectric block into a plurality of individual transducer elements after the matching layer has been formed. Cuts may be formed in the flex circuit substrate between adjacent transducer array elements to acoustically decouple adjacent elements. The flex circuit substrate and matching layers may have relatively high impedances to facilitate acoustic impedance matching between the sensor and the imaging environment.

### BACKGROUND OF THE INVENTION

This application relates to ultrasonic imaging devices such as ultrasonic imaging catheters and sensors and to methods for fabricating these devices.

Ultrasonic imaging techniques are often used to gather images during the diagnosis and treatment of medical conditions. An ultrasonic imaging catheter may be used to gather images from within a patient's body. During percutaneous transluminal coronary angioplasty procedures, for example, images may be acquired from within the blood vessels of a cardiac patient to help a physician to accurately place an expandable balloon.

In a typical ultrasound imaging catheter configuration, a piezoelectric ultrasound transducer array at the distal end of the catheter may be used to generate high-frequency acoustic signals that radiate towards the image target (e.g., a patient's blood vessel). The transducer array gathers corresponding reflected acoustic signals. Image processing techniques are used to convert the reflected acoustic signals into images for the physician.

It is an object of the present invention to provide improved ultrasonic imaging catheters and sensors and methods for fabricating such devices.

### SUMMARY OF THE INVENTION

This and other objects of the invention are accomplished in accordance with the principles of the invention by providing ultrasonic imaging catheters and sensors and methods for their fabrication.

An imaging catheter constructed in accordance with the invention may have a sensor at its distal tip. The sensor may have a transducer array formed from piezoelectric elements. During the manufacturing process, a uniform acoustic matching layer may be formed over the surface of all of the elements in the transducer array in parallel. These simultaneously-formed acoustic matching layer portions on the transducer array elements improve the performance of the transducer array when the imaging catheter is used to gather images of a suitable image target (e.g., the blood vessels or other body lumens of a patient). Specifically, the matching layer matches the acoustic impedance of the transducer elements to the surrounding medium (e.g., blood, tissue, etc.) by serving as an intermediate layer of intermediate impedance and suitable thickness. Thus, the matching layer may be formed using a material that has an acoustic impedance between that of the transducer elements and the body fluid or other substance in which the sensor is immersed during operation.

The sensor may have a flexible circuit (“flex circuit”) on which the transducer array elements are formed. The flex circuit substrate may help to match the acoustic impedance of the piezoelectric transducer elements to the acoustic impedance of the medium in which the sensor is immersed. To enhance the acoustic matching capabilities of the flex circuit, the flex circuit substrate may be formed using a flexible substrate material with a relatively high acoustic impedance for flexible polymeric materials. As an example, within the polyimide group of materials, Upilex has a higher acoustic impedance than Kapton. The flex circuit may have conductors to which the transducer array elements are electrically connected.

The transducer array elements may be mounted to the flex circuit in the form of a block of piezoelectric material that is subsequently divided to form the individual transducer array elements. During the process of dividing the transducer array into individual elements, the flex circuit that underlies the transducer array may be divided as well, so as to acoustically decouple the transducer array elements from adjacent elements. This is expected to improve imaging performance by reducing cross-talk.

Further features of the invention, its nature and various advantages will be more apparent from the accompanying drawings and the following detailed description of the preferred embodiments.

### BRIEF DESCRIPTION OF THE DRAWINGS

FIG. 1 is a perspective view of an illustrative ultrasonic imaging catheter and accompanying image processing equipment in accordance with the present invention.

FIG. 2 is a perspective view of the distal tip of an illustrative support lumen that may be used in the core of an ultrasonic imaging catheter device in accordance with the present invention.

FIG. 3 is a perspective view of the distal tip of another illustrative support lumen that may be used in the core of an ultrasonic imaging catheter device in accordance with the present invention.

FIG. 4 is a perspective view of a transducer array and integrated circuits that have been formed on a flex circuit in accordance with the present invention.

FIG. 5 is a cross-sectional side view of a portion of an illustrative ultrasonic imaging catheter in the vicinity of the transducer array and accompanying integrated circuits in accordance with the present invention.

FIG. 6 is a cross-sectional view of the illustrative catheter of FIG. 5 taken along the line 6-6 in accordance with the present invention.

FIG. 7 is a perspective view of an illustrative transducer array element showing the placement of the transducer electrodes in accordance with the present invention.

FIG. 8a is a cross-sectional end view of the transducer array element of FIG. 7 mounted in an illustrative fashion to a flex circuit in accordance with the present invention.

FIG. 8b is a perspective view of a portion of a flex circuit and piezoelectric transducer prior to using a conductive fillet to connect the transducer electrodes to electrical conductors on the flex circuit in accordance with the present invention.

FIG. 8c is a perspective view of a portion of a flex circuit and piezoelectric transducer after using a conductive fillet to connect the transducer electrodes to electrical conductors on the flex circuit in accordance with the present invention.

FIG. 9 is a cross-sectional view (in perspective) of an ultrasonic transducer array having multiple elements of the types shown in FIG. 7 and 8a after mounting to a flex circuit in accordance with the present invention.

FIGS. 10 and 11 are schematic views of illustrative template structures that may be used when fabricating transducer arrays in accordance with the present invention.

FIG. 12 is a side view of an illustrative template structure prior to insertion of a block of piezoelectric material for processing to form a transducer array in accordance with the present invention.

FIG. 13 is a side view similar to that of FIG. 12 showing how a piezoelectric block may be inserted into the template during the process of fabricating the transducer array in accordance with the present invention.

FIG. 14 is a side view of the template and piezoelectric block of FIG. 13 after the block and template have been covered by a flexible cover in accordance with the present invention.

FIG. 15 is a side view of the template structure after the flexible sheet of FIG. 14 has been removed in accordance with the present invention.

FIG. 16 is a side view of the template structure of FIG. 15 after a layer of acoustically matching material has been deposited on the piezoelectric block and planarized in accordance with the present invention.

FIG. 17 is a side view of the piezoelectric block after removal from the template structure of FIG. 16 in accordance with the present invention.

FIG. 18 is a flow chart of steps involved in fabricating an ultrasonic imaging sensor and catheter in accordance with the present invention.

FIG. 19 is a perspective view showing the end of a portion of an illustrative sensor in which transducer array elements have been acoustically decoupled from each other by forming cuts through flex circuit portions between adjacent elements in accordance with the present invention.

FIG. 20 is a perspective view of the distal portion of an illustrative ultrasonic imaging catheter showing (from an exterior perspective) the location of cuts made to acoustically isolate individual transducer array elements from each other in accordance with the present invention.

### DETAILED DESCRIPTION OF THE PREFERRED EMBODIMENTS

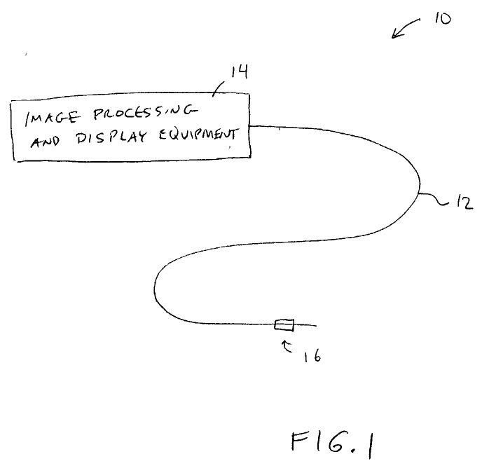

An illustrative catheter-based ultrasonic imaging system 10 in accordance with the invention is shown in FIG. 1. A catheter 12 with a ultrasonic sensor 16 may be used to gather images from locations inside a patient's body during surgical and diagnostic procedures. For example, catheter 12 may be used during percutaneous procedures such as cardiac surgery to gather images from inside a patient's blood vessels. The catheter may be used for other ultrasound applications if desired.

The catheter may have any suitable dimensions. For example, the catheter may have an overall length of about 100-200 cm. The distal portion of the catheter may have a length of about 35 cm and the proximal portion of the catheter may have a length of about 125 cm.

Image data from sensor 16 may be provided to image processing and display equipment 14 using cables within catheter 12. Equipment 14 may use ultrasound image processing techniques to process the image data and to display corresponding images on a display screen for a physician or other suitable user. Equipment 14 provides signals to sensor 16 that control the operation of sensor 16. For example, equipment 14 may provide drive signals for the transducer elements in sensor 16 that cause those elements to emit appropriate acoustic signals into the surrounding area of the patient's body. Appropriate acoustic signals may, for example, be acoustic pulses of approximately 25 ns in duration.

The peak drive voltage that is impressed upon the piezoelectric transducer elements to generate these pulses may be about 10 V. The acoustic echoes from the patient's body may induce voltages in the transducer elements on the order of 10-30 μV. Because the induced signals from reflections are typically several orders of magnitude lower than the drive signal, the acoustic impedance of the sensor is preferably matched to the surrounding environment. Individual transducer elements may also be acoustically isolated from each other to improve imaging performance.

Sensor 16 may be mounted on any suitable medical device such as a catheter of the type shown in FIG. 1 or a probe. For example, sensor 16 may be mounted on a catheter that contains a hollow tube. A guide wire that runs through the hollow tube may be used to assist in the placement of the catheter at a desired location within the patient's body. In another suitable arrangement, sensor 16 may be affixed to the end of a probe without a hollow tube that is manipulated at its proximal end by a physician or other suitable user. For clarity, the present invention will be describe primarily in the context of sensor arrangements involving catheters. This is, however, merely illustrative. Sensor 16 may be used with any suitable intraluminal and/or medical instrument or equipment.

Sensor 16 may be permanently affixed to a medical instrument or may be provided as part of a separate structural element that is removable from the instrument. For example, sensor 16 may be provided as part of an interchangeable catheter tip.

Additional components may be provided on catheter 12 if desired. For example, fiber-optic cables may be used to provide video imaging capabilities, expandable balloons may be used to deploy stents, atherectomy tools may be used to clear blockages, snares or probe tips may be provided for use during surgery, irrigation ports or guide wire passages may be provided, and temperature sensors, flow sensors (e.g., Doppler flow sensors), pressure sensors, and combinations of such sensors may be provided, etc. These are merely illustrative examples. Any suitable components may be included in catheter 12 if desired.

Sensor 16 may include a transducer array based on a number of individual transducer elements. Any suitable number of elements may be used (e.g., 16, 32, 64, 80, 128, 256, more than 256, less than 16, 32-256, etc.). The transducer elements may be formed of a piezoelectric material (e.g., a ceramic piezoelectric material, a polymeric piezoelectric material, a single crystal piezoelectric material, a composite piezoelectric material having ceramic particles in a matrix, etc.). When driven by suitable AC electrical signals, the transducer elements produce acoustic waves that radiate into the patient's body. These transducer elements (or a separate set of receiver transducer elements) are used to gather reflected acoustic waves and to convert those signals into acoustic energy.

In one suitable arrangement, sensor 16 includes a number of transducer elements that are formed on a flexible substrate that includes conductive electrodes for carrying electrical signals to and from the transducer elements. This type of substrate, which is referred to as a “flex circuit,” is relatively thin (e.g., 5 μm to 200 μm thick or any other suitable thickness) and may be rolled up or otherwise manipulated to form a cylinder that is affixed to the cylindrical core of the catheter 12 at an appropriate distal location as shown in FIG. 1. The flex circuit portion may be on the outermost portion of the sensor, so that during operation acoustic energy from the transducer array elements radiates radially outward through the flex circuit into the patient's body. The outer diameter of the flex circuit after it has been attached to the catheter may be on the order of 0.5 mm to 3 mm or any other suitable diameter.

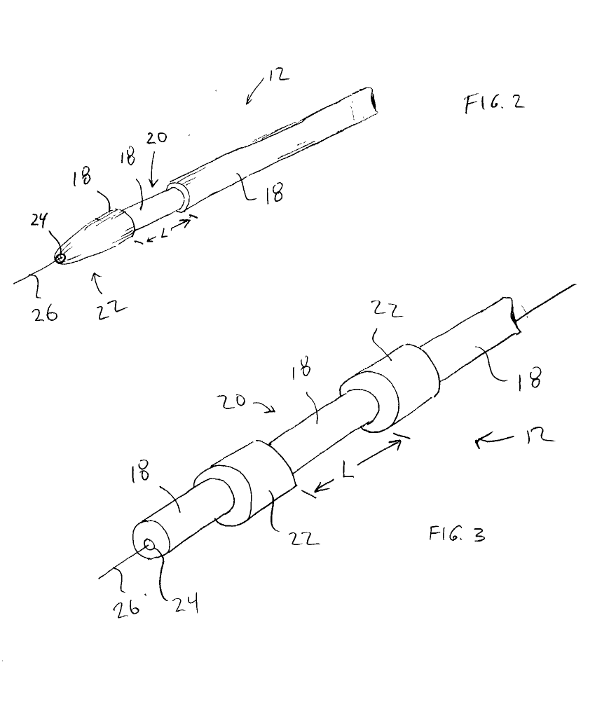

It may be desirable to place an acoustic backing layer between the catheter core and the transducer array elements to reduce acoustic interference in the form of spurious “ringing” signals reflected from the core into the transducer elements. The backing layer and rolled-up cylindrical transducer array may be mounted in a groove in the catheter core. Suitable catheter arrangements having a groove for accommodating the backing layer material and cylindrical sensor elements are shown in FIGS. 2 and 3.

In the arrangement of FIG. 2, catheter 12 has a main cylindrical core or body portion formed from a hollow or solid tube or a series of tubes or tube sections or other individual parts. The catheter core may be formed using metal, plastic, a combination of metal and plastic parts, or any other suitable material or combination of materials. All or a portion of the catheter (e.g., in the vicinity of the catheter tip) may be formed using a radiopaque material, e.g., a material that is visible during x-ray fluoroscopy, such as a metal alloy containing platinum or iridium. A typical hollow tube may have a diameter of approximately 0.024 inches and a wall thickness of 25 μm. At the distal tip of catheter 12, the elements of core 18 may be shaped to form a circumferential groove 20. Groove 20 is preferably of sufficient depth to accommodate all or at least some of the thickness of the backing layer material and the transducer array elements when the catheter is fully assembled. In the illustrative arrangement of FIG. 2, groove 20 is formed by forming an indentation with the components of catheter core 18 between a distal portion attached to the main length of the catheter and a tip portion 22. Tip 22 may be formed from the same material or materials as catheter core 18 or may be formed from a soft material that, in combination with the tapered shape of tip 22, facilitates insertion of catheter 12 into narrow lumens such as blood vessels. A guide wire lumen 24 may be provided in catheter 12 so that catheter 12 may be advanced over guide wire 26.

In the illustrative arrangement of FIG. 3, annular rings 22 have been used to form spacers that define a groove 20. Rings 22 may be formed of any suitable material. For example, rings 22 may be formed from rubies, which advantageously may be machined to tight tolerances. Other suitable materials that may be used include plastic, ceramic, metal, epoxy, composites of such materials, etc. Rings 22 may be affixed to core 18 using an adhesive such as a cyanoacrylate adhesive. Cyanoacrylate adhesive may also be used to affix other portions of core 18 to each other. For example, when core 18 is formed from nested or overlapping tubes, cyranoacrylate adhesive may be used to secure the tubes to each other.

As shown in FIGS. 2 and 3, a guide wire lumen 24 that runs longitudinally through the center of catheter 12 may be provided. This allows catheter 12 to be guided over a guide wire 26, which facilitates placement of catheter 12 during use in a patient.

The arrangements for defining the groove 20 on catheter core 18 that are shown in FIGS. 2 and 3 are merely illustrative. Any suitable arrangement may be used to define groove 20 if desired. The groove may be defined by forming an indentation in the tube or tubes or cylinder of material that is used to form the main body of catheter 12, may be formed by adding additional spacers, rings or other structures over the outer circumference of the core 18, may be formed by attaching a specially formed tip having an integral groove portion, or may be formed using other suitable approaches or combinations of such approaches.

As shown in FIGS. 2 and 3, groove 20 may have a length L (as measured along the longitudinal axis of catheter 12). The length L may be sufficient to accommodate the transducer array when the transducer array and other components on the flex circuit are rolled up to form a cylinder around the distal end of catheter 12. For example, if the corresponding dimension of the transducer array is 1 mm, then length L should be 1 mm plus a small clearance to allow the transducer array to be mounted in groove 20.

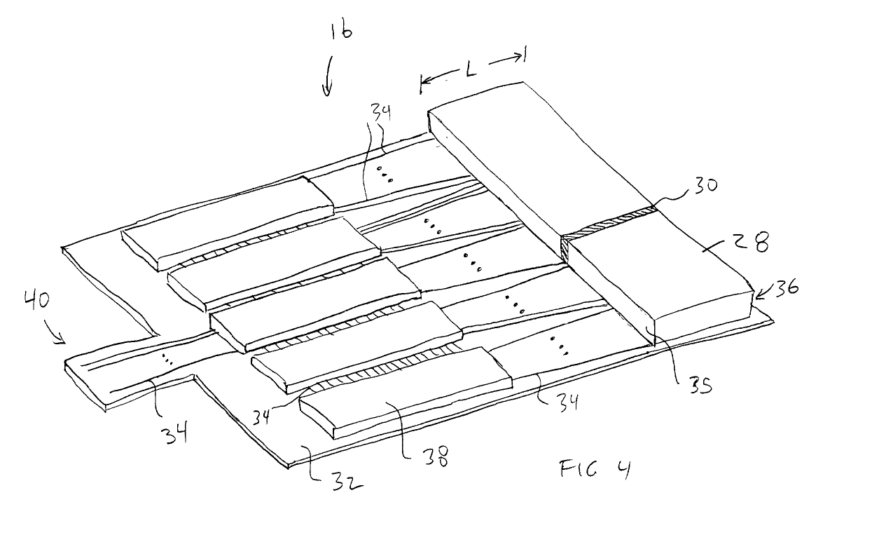

An illustrative sensor 16 having a transducer array 28 with a dimension L for mounting in a groove 20 of the type shown in FIGS. 2 and 3 is shown in FIG. 4. Transducer array 28 may have a number of individual elements, each of which is aligned in parallel with the illustrative element 30 shown in FIG. 4. The transducer array 28 is mounted on a flex circuit 32. The flex circuit may be formed from a flexible substrate material such as polyimide, which is electrically insulating. If desired, the flex circuit may be formed from a substance having a relatively high acoustical impedance for flexible polymeric materials. Within the polyimide group of materials, Upilex has a higher acoustic impedance than Kapton. With one suitable arrangement, the flex circuit may be formed form a substance that is flexible and that has an acoustic impedance of at least 3.5 MRayls or an acoustic impedance in the range of 3.5 to 4.5 MRayls. A suitable flex circuit substrate material that has an acoustic impedance of about 4 MRayls is available under the trade name Upilex-S, from Ube Industries, Inc. of Yamaguchi, Japan. Using a flex circuit with an acoustic impedance of 3.5 to 4.5 MRayls may significantly improve the acoustic impedance matching between sensor 16 and the medium in which catheter 12 is immersed (typically a patient's blood). Such acoustic impedance matching is expected to improve the performance of the sensor 16.

Electrical conductors 34 are formed on the surface of the flex circuit substrate. The electrical conductors may be formed, for example, from a malleable metal such as gold. A suitable adhesion layer such as a thin layer of chromium may be used to facilitate adhesion of the gold or other conductor material to the substrate. Metal layers may be deposited by sputtering, evaporation, or any other suitable technique. Wet or dry etching or other suitable patterning techniques may be used to pattern the deposited metal to form electrical conductors 34.

Each transducer element 30 may have two opposing electrodes. The main portion of the electrodes is located on the upper and lower surfaces of the transducer array when the array is oriented as shown in FIG. 4. Smaller portions of the electrodes extend over the ends 35 and 36 of the elements 30 in transducer array 28. Electrical signals may be conducted between the conductors 34 and the main portions of the electrodes by forming electrical contacts between the conductors 34 and the end portions 35 and 36.

By connecting the electrodes on each transducer element 30 to corresponding conductors 34, drive signals for the transducer elements 30 may be conveyed to the elements 30. Similarly, electrical signals that are produced by the elements 30 when reflected acoustic waves are detected by elements 30 may be conveyed from the elements.

In some transducer arrays (e.g., arrays with 64 elements or more), there may be so many conductors 34 that it is cumbersome to route all of these conductor lines to equipment 14 in a single cable along the length of the catheter 12. Accordingly, electrical multiplexer circuits 38 (e.g., time-division multiplexing circuits or other suitable multiplexing circuits) may be used to reduce the relatively large number of conductors 34 that are directly connected to transducer array 28 into a smaller number of conductors 34 at the input/output 40. The conductors at input/output 40 may be soldered, welded, or otherwise electrically connected to wires in a suitable cable that runs along the length of catheter 12 to equipment 14. If desired, circuits 38 may include drive circuitry for generating drive signals and/or preprocessing circuitry for at least partially processing the electrical signals that are produced when the transducer elements 30 in array 28 are used to detect acoustical information.

After circuits 38 and transducer array 28 have been mounted on flex circuit 32, as shown in FIG. 4, the flex circuit and mounted components may be formed into a cylindrical shape and attached to the distal section of catheter 12, so that array 28 protrudes into groove 20 (see FIGS. 2 and 3). A cross-sectional side view of an illustrative catheter 12 after the flex circuit and components of FIG. 4 have been attached to the catheter is shown in FIG. 5.

As shown in FIG. 5, the ends 35 and 36 of array 28 are held in place between the inner ends of groove 20 (defined by the circumferential inner end faces of annular spacer rings 22 in the embodiment of FIG. 5). Integrated circuits 38 may surround the catheter core as shown in FIG. 5.

The body of catheter 12 may have a guide wire tube 106 (e.g., a high-density polyethelyne tube) surrounded by an optional outer tube 108 (e.g., a medium-density polyethylene tube) and a corresponding tube extension 110. Circuits 38 and array 28 may be wrapped around marker tube 112 and backing layer 46. If five circuits 38 are involved, the cross-section of the circuits will form a pentagon. If four circuits 38 are involved, the cross-sectional shape will be square. Other numbers of circuits 38 may be used if desired.

At the input/output 40 of the flex circuit, cable wires 42 may be soldered, welded, or otherwise electrically connected to the conductors 34 on the flex circuit. The catheter 12 may have a longitudinal lumen through which the cable 42 passes to equipment 14 (FIG. 1). Plastic tube 118 may be affixed to tube extension 110 using cyanoacrylate adhesive 120. Cyanoacrylate adhesive may also be used as the adhesive 122 for affixing outer tube 108 and tube extension 110 to marker tube 112. An ultraviolet-curable adhesive 124 may be used to seal and attach the sensor 16 to the rest of catheter 12.

Stiffening member 126 may be used to stiffen the proximal portion of catheter 12.

This is merely one suitable arrangement for mounting the flex circuit and components such as circuits 38 and transducer array 28 to the catheter 12. Any suitable arrangement may be used if desired. For example, separate tubes may be provided as unitary structures. Single tubes or structures may be provided in the form of individual parts that are affixed using adhesives or other suitable arrangements. Different types of adhesives and tubing may be used, etc.

The backing layer 46 that is used to support the transducer array 28 preferably has a relatively high acoustic attenuation, so that acoustic signals propagating radially inward to the center of the catheter core from transducer array 28 are absorbed. The impedance of layer 46 is preferably above 3 MRayls, and even more preferably above 4 MRayls (e.g., 4.2 MRayls). Suppressing spurious acoustic signals in this way helps to reduce ringing and improves the signal to noise ratio of the sensor 16. Any suitable material may be used for backing layer 46. For example, backing layer 46 may be formed from a mixture of epoxy and hollow microspheres or any other suitable material that has a high acoustic absorbance. It may also be advantageous for the backing layer to be formed from an adhesive so that it may reduce “ringing,” as well as attach/secure the transducer array to the catheter body.

During the process of forming transducer array 28, an acoustic matching layer may be formed on each of the array elements 30. The matching layer may be formed, for example, from a 20-30 μm thick layer of material having an acoustic impedance that is preferably in the range of 5-12 MRayls or more preferably in the range of 8-10 MRayls. An illustrative matching layer material that may be used is Eccosorb™, available from Emerson & Cuming Microwave Products, Inc. of Randolph, Mass.

The matching layer may be disposed between transducer array elements 30 and flex circuit substrate 32. During operation, acoustical signals are transmitted from the transducer array elements through the matching layer and the flex circuit substrate. The thicknesses (totaling about a quarter-wavelength of the acoustical signal wavelength) and acoustical impedances of the matching layer and flex circuit substrate may be selected to provide good acoustical impedance matching between transducer array 28 and the surrounding tissue or other substances in the patient's body that are being imaged by the ultrasound sensor 16.

The quality of sensor 16 may be characterized in terms of performance metrics such as sensitivity (efficiency), ringdown (the ability of the transducer to stop vibrating immediately after an acoustic pulse has been produced by the transducer array), bandwidth (the ability of the transducer to launch and receive a wide frequency range of acoustic signals), and cross-talk (the relative electrical/acoustic isolation of individual elements of the transducer array from adjacent elements).

The use of an acoustic matching layer on transducer array elements 30 improves the ringdown performance of the sensor 16 significantly as compared to configurations in which acoustic matching capabilities are only provided using the flex circuit substrate material itself. This is due primarily to the higher acoustic impedance properties of the matching layer compared to those available from suitable flexible circuit substrates.

The acoustic matching layer is used to form an acoustical antireflection coating that helps couple acoustical signals into and out of the transducer array 28. Ideally, the acoustic matching layer would have an acoustic impedance roughly equal to the geometric mean of the acoustic impedance of the piezoelectric material of the transducer array 28 (about 31 MRayls) and the medium in which the catheter is immersed (typically a patient's blood or other body fluid, which has an acoustic impedance of about 1.5 MRayls). The geometric mean of these two values (given by the square root of their product) is about 6.8 MRayls.

The materials that are most suitable for the substrate of flex circuit 32 are drawn polymer films such as polyimide (Upilex or Kapton). Such films are flexible enough to roll up into the cylindrical shape needed for sensor 16 after the electrical conductors 34 have been formed and the components such as circuits 38 and array elements 30 have been mounted on the flex circuit 32. However, such polyimide-like films typically have acoustical impedances of about 3.2 MRayls or less, which is significantly below the optimum value of about 6.8 MRayls. By using a matching layer with an acoustic impedance close to 6.8 MRayls (e.g., 5-12 or 6-8 MRayls), the acoustic matching between the transducer array and the medium in which the catheter is operating (e.g., blood) is improved, and ringdown (due to reflected energy at the interface between the device and the medium in which it is operating) is significantly improved.

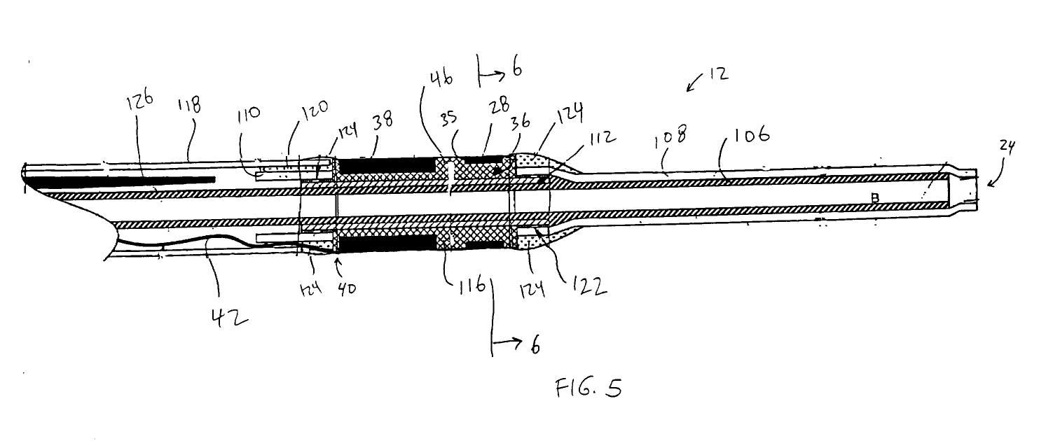

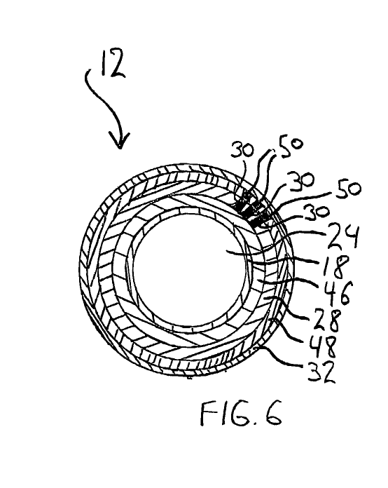

To ensure a high level of uniformity in the matching layer thickness and to improve the efficiency of the manufacturing process, the matching layer for all of the elements 30 in array 28 may be deposited and planarized in parallel. The acoustic matching layer and the other portions of the catheter tip are shown in cross section in FIG. 6 (taken along the line 6-6 in FIG. 5).

As shown in FIG. 6, catheter core 18 may have a lumen 24 in which a guide wire may be disposed during use of the catheter. Core 18 may be solid or may be formed using a hollow tube (e.g., a hollow plastic tube). If core 18 is a hollow tube, lumen 24 may be the same size as the inner bore of the tube or may be provided by nesting a separate hollow tube within the catheter tube. These are merely illustrative configurations. Any suitable configuration may be used if desired.

A backing layer 46 that is highly absorbing to acoustic waves may be provided on the outer surface of the hollow tube or cylinder that forms core 18. The transducer array 28 may be attached to core 18 after backing layer 46. Matching layer 48 is disposed between the flex circuit 32 and the array 28. The array 28 is shown as forming an annular ring in the drawing of FIG. 6, but is actually composed of many individual transducer array elements 30 (three of which are shown in FIG. 6). Similarly, the matching layer 48 is shown as forming a continuous layer of material in the drawing, but actually lies on top of each of the transducer array elements 30, as indicated by the three illustrative matching layer portions 50. The drawing of FIG. 6 is not to scale.

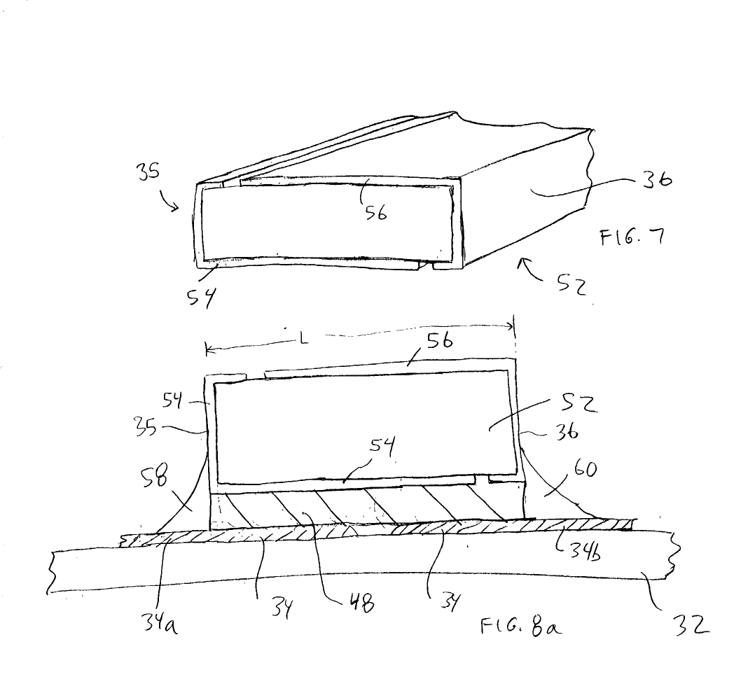

As described in connection with FIG. 4, the electrodes on each transducer element are electrically connected to corresponding electrical conductors 34 on flex circuit 32 prior to installation of the transducer elements in the catheter. A perspective view of a block of piezoelectric material 52 that is to be used to form transducer array 28 is shown in FIG. 7. The piezoelectric block 52 of FIG. 7 has not yet been divided into individual elements 30. Electrodes 54 and 56 may be formed on block 52 using sputtering, evaporation, or other suitable deposition techniques and wet or dry etching or shadow masking or other suitable pattering techniques. These are merely illustrative methods for forming electrodes 54 and 56. Any suitable techniques may be used to form electrodes 54 and 56 if desired. Electrodes 54 and 56 may be formed from gold (e.g., with an underlying adhesion layer of chromium or the like) or any other suitable metal or conductor.

When piezoelectric block 52 is installed on flex circuit 32, the end faces 35 and 36 are electrically connected to conductors 34 on the flex circuit substrate. One suitable technique for electrically connecting end faces 35 and 36 to conductors 34 is to use conductive portions such as conductive fillets 58 and 60 (e.g., silver paste fillets), as shown in FIG. 8a. (FIGS. 7 and 8a and the other FIGS. are not drawn to scale. For example, matching layer 48 may be about 20-30 microns thick and piezoelectric block 52 may be about 66-77 microns thick and 500-1000 microns in length L—i.e., a 14× aspect ratio.)

Conductive portion 58 electrically connects end face portion 35 of electrode 54 to portion 34a of conductors 34. Conductive portion 60 electrically connects end face portion 36 of electrode 56 to portion 34b of conductors 34. Conductors 34 carry signals (drive signals for the array elements or acoustic echo signals that have been converted by the array elements into electrical signals) between the piezoelectric elements and the electronics in circuits 38 and equipment 14.

As shown in the lower portion of FIGS. 8a and 8b, matching layer 48 lies under lower electrode 54, between piezoelectric block 52 and the substrate of flex circuit 32. FIG. 8c shows how fillet 58 is used to form electrical contact with end face 35 and conductor 34.

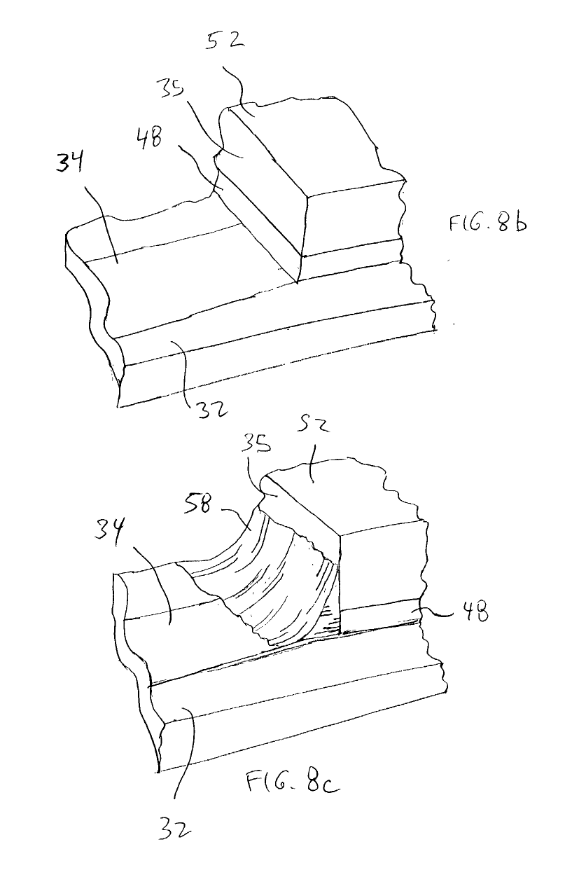

After the electrodes on block 52 have been electrically connected to conductors 34 as shown in FIG. 8a, 8b, and 8c, the piezoelectric block 52 may be divided into individual transducer elements 30, as shown in FIG. 9. Block 52 may be divided into elements 30 by sawing block 52 (e.g., to leave spaces such as kerfs 62 between each respective pair of elements 30), by scoring block 52 (e.g., using a knife edge), by laser-cutting or water-jet-cutting of block 52, or by using any other suitable cutting or dicing scheme.

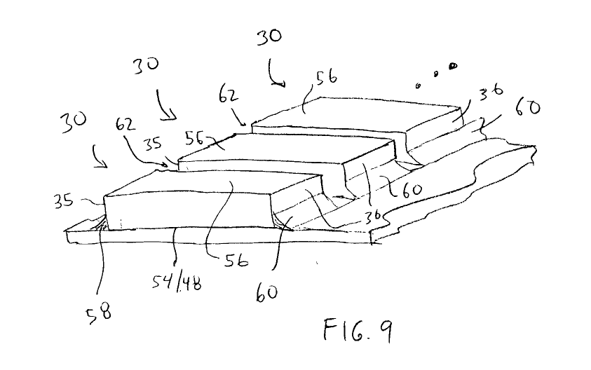

The matching layer portions on each transducer element 30 may be formed simultaneously using any suitable technique. One illustrative approach for forming matching layer 48 on piezoelectric block 52 involves using template structures such as template structures 64 of FIGS. 10 and 11. During the manufacturing process, rectangular blocks of piezoelectric material such as block 52 of FIG. 7 may be inserted into the recessed holes 68 defined in template layer 66 of structure 64. The matching layer may be deposited over the piezoelectric blocks. The sides of the holes 68 help to prevent the matching layer material from coating the side walls of block 52 and ends such as ends 35 and 36. The template structure 64 of FIG. 11 has larger holes 68, which may be used to protect the walls of larger blocks of piezoelectric material. Such larger blocks may then be cut down to form smaller, array-sized blocks of the type handled by holes 68 of FIG. 10. In the illustrative examples of FIGS. 10 and 11, only a relatively small number of holes 68 are shown. In practice, larger number of holes 68 may be provided (e.g., 50-100 or more) to improve the throughput and economies of scale involved in processing multiple piezoelectric blocks at a time.

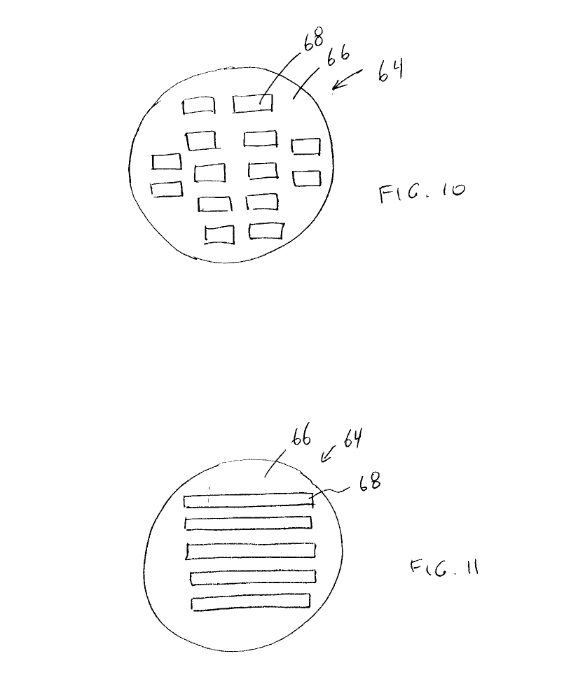

FIGS. 12-17 show side views of the template structure and piezoelectric block 52 during processing. As shown in FIG. 12, hole 68 may have a length L that is substantially the same as the dimension L of the piezoelectric block 52. Template material 66 may be wax or plastic or any other suitable material for protecting the sidewalls of piezoelectric block 52. Preferably, template material 66 flows when heated. The template material 66 may have a thickness T that is about the same as the thickness of the piezoelectric block. For example, the thickness T may be the same as the thickness of the piezoelectric block 52 or thickness T may be thinner than the piezoelectric block thickness by up to about 10 μm. Using a template 66 that is equal or less than the piezoelectric block in thickness helps to ensure that the tops of the blocks are well sealed during subsequent top-layer masking operations. Template material 66 may be supported by a substrate 70 (e.g., a stainless steel substrate or carrier).

As shown in FIG. 13, each hole 68 in template structure 64 may be filled with a block 52 with an upper electrode 56 and a lower electrode 54.

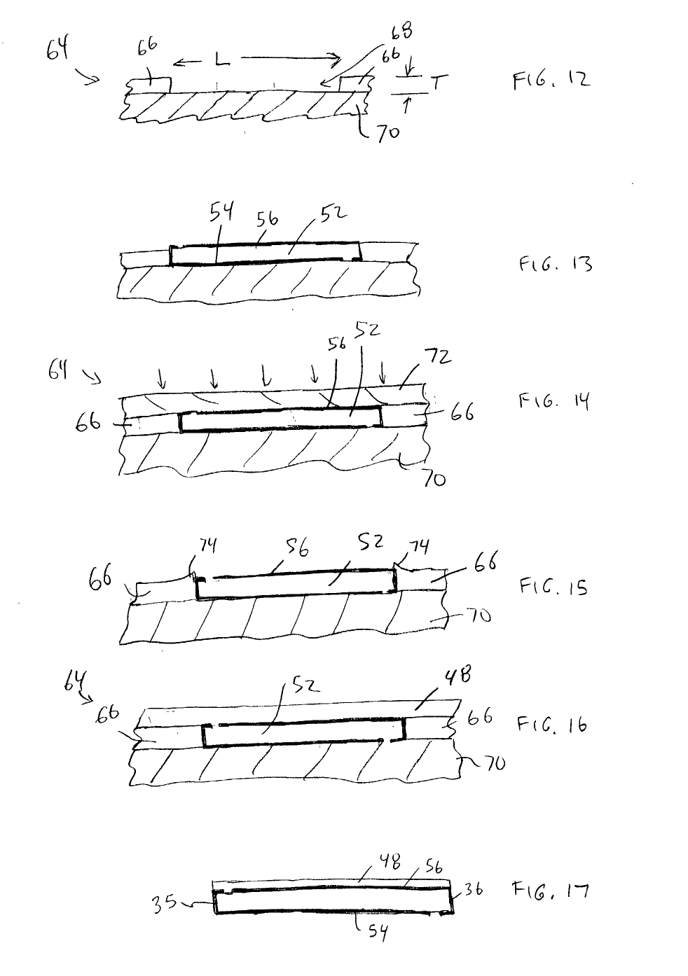

After block 52 has been inserted into the hole defined by the template 66, a flexible masking member 72 (e.g., a flexible silicone cover) may be pressed against the upper surface of the piezoelectric blocks 52 and template portions 66 as shown in FIG. 14. The template structure and blocks 52 may then be heated. This causes the template material to flow. As the plastic or wax or other template material of template layer 66 flows, it coats the side walls of blocks 52. However, the flexible cover 72 seals off the top layer of block 52, thereby preventing the wax or other template material from flowing over top electrode 56. By protecting top electrode 56 in this way, subsequent cleaning operations may be minimized.

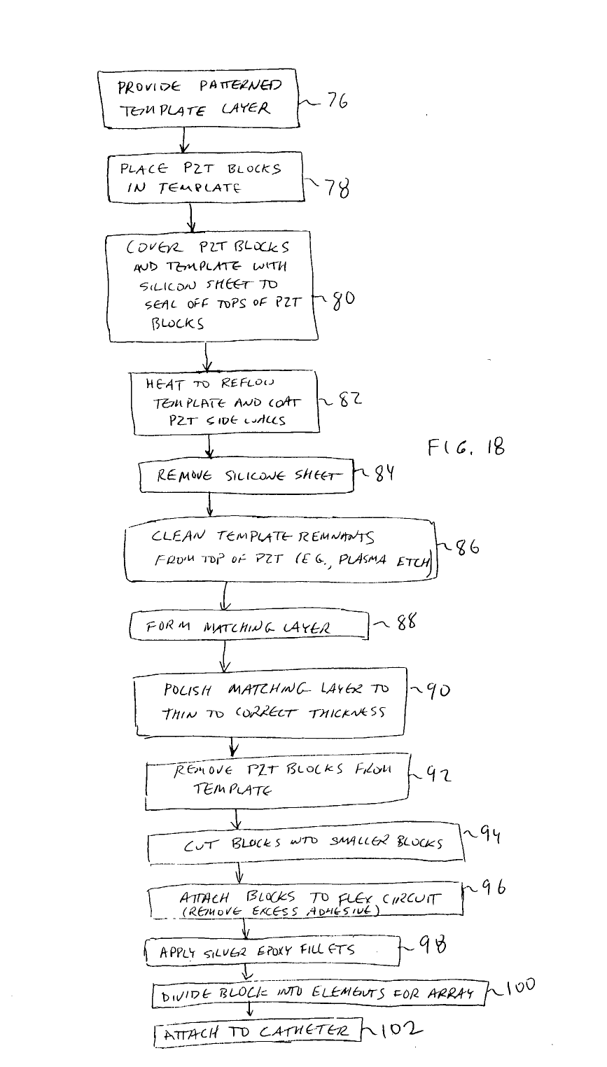

After the cover 72 has been removed and the template cooled (optionally, the template may be cooled prior to removal of cover 72), the template layer may appear as shown in FIG. 15. Cusps 74 and other stray template material may be removed from the top electrode 56 by plasma etching (e.g., in an oxygen plasma) or any other suitable cleaning procedure.

As an alternative to flowing, template 66 may be provided with very tight tolerances such that the template is substantially the same width L as blocks 52. This allows application of a matching layer to top electrode 56, while ensuring that the matching layer does not contact the side wall portions of electrodes 54 and 56 of blocks 52. As with the flowing approach, the height of the template may be modified and/or stray template material may be removed from the top electrode 56 by plasma etching (e.g., in an oxygen plasma) or any other suitable cleaning procedure.

The matching layer 48 may be formed by spreading a suitable matching layer material on top of the cleaned structures, by curing the matching layer material, and by polishing the cured matching layer (if desired). Suitable matching layer materials such as Eccosorb typically are formed from precursors that have a paste-like consistency. After curing, the matching layer becomes solid. Mechanical polishing, chemical-mechanical polishing, or any other suitable polishing or planarizing technique may be used to planarize the matching layer portions on top of each of the piezoelectric blocks 52 in the template structure 64 in parallel. A side view showing how matching layer 48 may be formed on top of block 52 and template 66 is shown in FIG. 16.

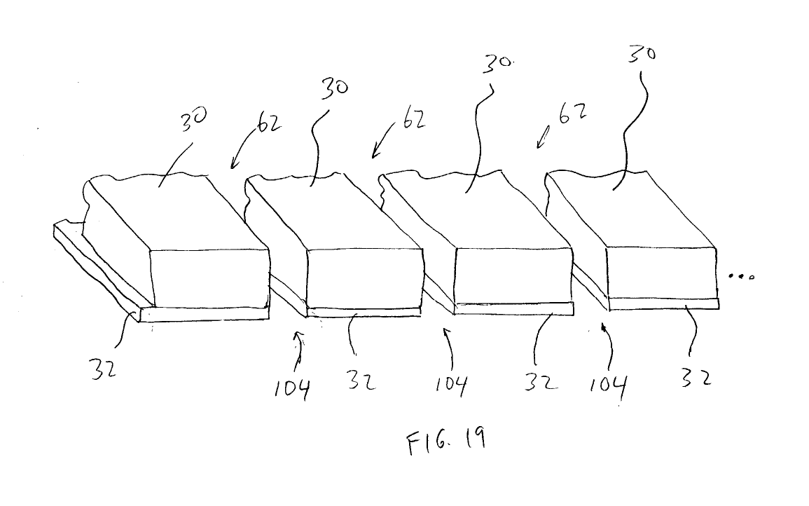

After the matching layer 48 has been polished and blocks 52 removed from the template and cleaned in a solvent (if desired) to remove excess template material from the side walls, each block 52 (now coated with matching layer 48 as shown in FIG. 17) is ready to be affixed to flex circuit 32 and electrically interconnected with conductors 32. Blocks 52 may be removed from template 66 by a variety of techniques, including laser or mechanical cutting. Alternatively, the template may be thermally cooled and fractured along interfaces with blocks 52. Further, the template material, e.g., wax, may be melted to allow easy removal of the blocks from the template. These are merely illustrative examples. Any suitable technique for removing blocks 52 having matching layers 48 from template 66 may be used if desired.

During the process of coating blocks 52 with matching layer 48, the matching layer on each of multiple blocks 52 (e.g., 50-100 blocks in structure 64) are formed simultaneously (both during the deposition and curing phase and during the polishing phase). Preferably, matching layer 48 is segmented from a sheet covering multiple blocks 52 into individual portions that individually cover the surface of each block 52 during removal of the blocks from the template. Such segmentation may be achieved by cutting, cracking, etc.

Moreover, each of the transducer elements 30 that is created when a given piezoelectric block 52 is divided up has a portion of the simultaneously-formed matching layer 48, which is also divided up during the dicing of block 52 into elements 30. Because they are produced in parallel, the simultaneously-formed matching layer portions on the transducer elements are uniform and economical to manufacture.

Illustrative steps involved in fabricating an ultrasonic imaging catheter 12 having simultaneously-formed matching layer portions 48 on each of the transducer elements 30 in the array 28 are shown in FIG. 18. At step 76, the patterned template structure 64 may be provided (e.g., by forming a patterned wax or plastic template 66 on a stainless steel carrier 70).

At step 78, piezoelectric blocks 52 may be placed into each of the holes 68 in the patterned template structure 64. The piezoelectric blocks 52 may be formed from any suitable piezoelectric material such as lead zirconate titonate composites. Electrodes 54 and 56 (including end-wall electrode portions 35 and 36) are preferably formed on the blocks 52 before the blocks are inserted into the holes 68.

At step 80, the piezoelectric blocks 52 may be covered with a suitable flexible mask (e.g., a silicone cover sheet). The cover may be pressed against the top surface of the blocks with sufficient force to seal off the tops of the blocks (and therefore electrodes 56).

At step 82, the template material (and the blocks 52) may be heated. This causes the template material to flow and coat the sides of the blocks 52.

At step 84, the suitable flexible mask may be removed.

Excess material, e.g., wax or plastic, may be removed by cleaning the blocks 52 using a plasma etch or other suitable cleaning technique at step 86.

At step 88, the matching layer may be formed simultaneously on all of the blocks 52 in the template structure 64 and on all of the portions of each block 52 that will later become individual transducer array elements 30. The matching layer may be deposited by applying uncured matching layer paste to the surface of the blocks 52 in template structure 64 and by applying heat and/or exposing the paste to air or any other suitable ambient environment to cure the paste.

At step 90, the cured matching layer 48 may be polished or otherwise planarized.

At step 92, the piezoelectric blocks 52 may be removed from the template structure 64 (e.g., by prying each block 52 from the structure 64 using a tool such as a knife, by flexing the substrate, by cooling the substrate and template structure 64 sufficiently to make the wax or plastic of the template 66 brittle enough to crack along the seams between the template 66 and the sides of the blocks 52 under pressure, by laser cutting, or using any other suitable technique). The sides of the blocks 52 will generally be clean when removed from the template, but an additional solvent cleaning step or other suitable cleaning operation may be used to further clean the template material from the blocks (and from the electrodes on the blocks) if desired.

If a template structure 64 with larger-sized holes such as holes 68 of FIG. 11 is being used, the larger sized piezoelectric blocks 52 may be cut into array-sized blocks at step 94.

At step 96, an individual block 52 may be attached to flex circuit 32 (e.g., using a suitable adhesive).

At step 98, conductive fillets or portions 58 and 60 may be used to interconnect the ends 35 and 36 of the electrodes 54 and 56 to the conductors 34 of flex circuit 32 (e.g., by applying silver paste along the interface between the ends of block 52 and the flex circuit).

At step 100, the block 52 may be diced, sawed or otherwise cut or divided into individual transducer array elements 30. Dividing block 52 into elements 30 also divides simultaneously-formed matching layer 48, such that each element 30 has an individual portion of matching layer 48.

If desired, step 100 may also involve coating the back of the flex circuit (at least in the vicinity of array 28) with a stabilizing layer of photoresist (e.g., polymethylmethacrylate or PMMA) or other suitable material, such as Nitto tape. The stabilizing layer is preferably temporary and may be used as a sacrificial layer to facilitate dicing and/or formation of the flex circuit into a cylinder. The dicing process (or other suitable cutting process) may then be used to cut through both the piezoelectric block 52 and, optionally, the underlying flex circuit under kerfs 62 (FIG. 9).

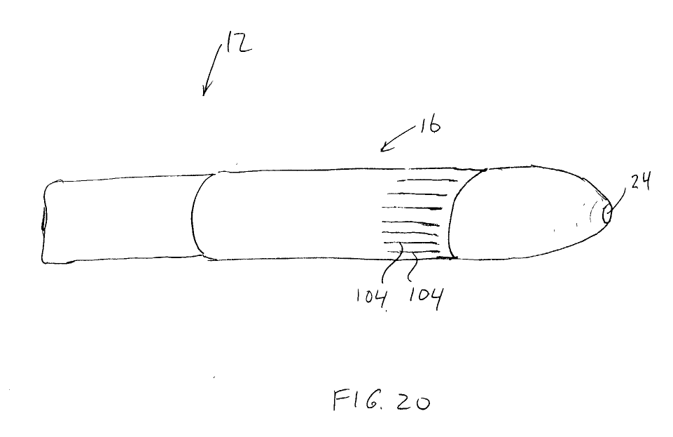

The cutting process optionally forms cuts 104 through all or some of the flex circuit 32 between adjacent transducer elements 30, as shown in FIG. 19. The drawing of FIG. 19 shows a sectional view (i.e., a cross-section across the middle of the transducer array) taken of the flex circuit 32 in a flattened condition, before being rolled up to form the cylindrical flex circuit shape of the sensor 16 that is used during operation of ultrasonic imaging catheter 12. The drawing of FIG. 20 shows, from an exterior perspective, how longitudinal cuts 104 (cuts parallel to the longitudinal axes of the sensor and catheter) are located (e.g., evenly-spaced) around the circumference of the distal end of the cylindrical rolled-up flex circuit that forms sensor 16.

The cuts 104 (which may be either downward extensions of kerfs 62 or separate cuts) help to isolate array elements 30 from each other. The cuts may extend from the edge of flex circuit 32 so as to leave each array element 30 mounted on its own piece of substrate material in a diving-board fashion. Alternatively, the ends of the “diving boards” may be left connected to each other by making cuts 104 through only the middle portions of the flex circuit 32 (i.e., the portion of flex circuit 32 between ends 36 and 36).

The underlying temporary stabilizing layer, which, by virtue of covering cuts 104, may facilitate the holding of the flex circuit 32 in the vacuum chucks used during sawing, may be removed after cutting is complete.

When the optional flex circuit cutting process is used during the process of dividing the array 28 into transducer array elements 30, the transducer array elements become more acoustically decoupled from each other. This decoupling may increase the width of the acoustic beam profile associated with each transducer array element 30.

Imaging quality is improved when each transducer element operates relatively independently and has a fairly wide associated beam profile. When the transducer array elements produce overly-narrow beams, the beams cannot be combined properly. This may make it difficult or impossible to sweep the combined beam through as wide an angle as is desired during the imaging process. By cutting completely through the portions of the flex circuit that lie between adjacent transducer array elements 30, the transducer elements produce wider beams and are less likely to induce undesirable vibrations in adjacent elements.

In FIG. 19, flex circuit 32 is shown cut into multiple pieces. However, as described above and as shown in FIG. 20, cuts 104 may be formed (during step 100 of FIG. 18) as local slits in the flex circuit in the vicinity of array elements 30, such that flex circuit 32 remains one piece distal of the array 28. Furthermore, cuts 104 may be filled with an epoxy or adhesive having a substantial acoustic mismatch to adjacent transducer elements 30. The adhesive may comprise, for example, a UV-cured or heat-cured epoxy. The epoxy facilitates formation of the flex circuit into a cylinder and provides a fluid seal between elements 30 of the transducer array, while still providing acoustic decoupling between respective array elements.

The performance of the ultrasonic imaging catheter 12 may be improved by using the simultaneously-formed acoustic matching layer portions on the elements 30 (e.g., the matching layer portions of Eccosorb formed by parallel-processing the array elements while still in the form of a unitary piezoelectric block), by using the high-acoustic-impedance flex circuit substrate (e.g., the substrate formed of Upilex-S, Kapton, or other material having an acoustic impedance in the range of 3.5-4.5 MRayls or other suitable range), by isolating adjacent array element 30 by cutting through the underlying flex circuit that lies between adjacent elements, or by using these design approaches in any suitable combination (e.g., using only one of these approaches or using any two of these approaches). Moreover, these aspects of the invention may be used in any suitable combination with the various other embodiments of the invention described above.

It will be understood that the foregoing is merely illustrative of the principles of this invention, and that various modifications can be made by those skilled in the art without departing from the scope and spirit of the invention.

The invention claimed is:

1. A sensor for(an ultrasonic imaging device operated in a medium having an acoustic impedance, comprising:
a flex circuit having a flex circuit substrate and a plurality of electrical conductors formed on the flex circuit substrate; and
an array of individual piezoelectric transducer array elements arranged around the flex circuit, wherein the transducer array elements have simultaneously-formed acoustic matching layer portions that help to match the acoustic impedance of the transducer array elements to the acoustic impedance of the medium, wherein the simultaneously-formed acoustic matching layer portions are disposed between the transducer array elements and the flex circuit substrate.

2. The sensor defined in claim 1 wherein the matching layer comprises a material having an acoustic impedance in the range of 5-12 MRayls.

3. The sensor defined in claim 1 wherein the flex circuit substrate has an acoustic impedance in the range of 3.5-4.5 MRayls.

4. The sensor defined in claim 1 wherein the transducer array elements are acoustically decoupled from each other by cuts formed through the flex circuit substrate between adjacent transducer array elements.

5. The sensor defined in claim 1 wherein a plurality of integrated circuits are mounted on the flex circuit.

6. The sensor defined in claim 1 wherein the flex circuit substrate comprises a flexible film having an acoustic impedance of at least 3.5 MRayls and the transducer array elements are acoustically decoupled from each other by cuts formed through the flex circuit substrate between adjacent transducer array elements.

7. The sensor defined in claim 1 wherein there are between 32 and 128 transducer array elements mounted to the flex circuit.

8. The sensor defined in claim 1 wherein there are electrodes on each of the piezoelectric transducer array elements, and wherein the electrodes are connected to the electrical conductors on the flex circuit using conductive fillets.

9. An ultrasonic imaging catheter sensor, comprising:
a flex circuit having a flex circuit substrate, wherein there are a plurality of electrical conductors on the flex circuit substrate; and
an array of individual piezoelectric transducer array elements arranged around the flex circuit, wherein the transducer array elements are separated by longitudinal cuts in the flex circuit substrate between adjacent transducer array elements.

10. The sensor defined in claim 9 wherein the transducer array elements are separated by kerfs and wherein the cuts are extensions of the kerfs.

11. The sensor defined in claim 9 wherein the flex circuit substrate comprises a material having an acoustic impedance in the range of 3.5-4.5 MRayls.

12. The sensor defined in claim 9 further comprising a matching layer of a material having an acoustic impedance in the range of 5-12 MRayls, wherein the matching layer is disposed on the transducer array elements between the transducer array elements and the substrate of the flex circuit.

13. A method of forming a flex-circuit sensor having multiple piezoelectric transducer array elements for an ultrasound imaging catheter, comprising:
placing a plurality of piezoelectric blocks in a template;
heating the template to cause the template to flow and cover the sides of the piezoelectric blocks;
forming a matching layer of a material having an acoustic impedance of 5-12 MRayls on the piezoelectric blocks after the sides have been covered;
mounting one of the piezoelectric blocks on a flex circuit so that the matching layer is between the piezoelectric block and the flex circuit; and
dividing the mounted piezoelectric block into individual piezoelectric transducer array elements.

14. The method defined in claim 13 further comprising shaping the flex circuit into a cylinder so that the individual piezoelectric transducer array elements form a substantially cylindrical ultrasound sensor array.

15. The method defined in claim 13 further comprising covering the tops of the piezoelectric blocks with a flexible cover to prevent the template from coating the tops of the piezoelectric blocks during heating.

16. The method defined in claim 13 further comprising polishing the matching layer.

17. The method defined in claim 13 further comprising forming cuts through the flex circuit between adjacent piezoelectric transducer elements.

18. The method defined in claim 13 further comprising:
placing a temporary stabilizing layer of material on the flex circuit before the formation of cuts through the flex circuit between adjacent piezoelectric transducer elements;
forming the cuts through the flex circuit between adjacent piezoelectric transducer elements; and
removing the temporary stabilizing layer after the cuts have been formed.

19. The method defined in claim 18 wherein the temporary stabilizing layer of material comprises a photoresist layer, the method further comprising holding the photoresist layer using a vacuum chuck.

20. The method defined in claim 13 further comprising mounting the piezoelectric block on a flex circuit having an acoustic impedance in the range of 3.5-4.5 MRayls.

21. The method defined in claim 13 wherein forming the matching layer comprises forming a matching layer having an acoustic impedance of 6-8 MRayls on the piezoelectric blocks.
